(19) United States
(12) Patent Application Publication
(10) Pub. No.: US 2021/0169706 A1
(43) Pub. Date: Jun. 10, 2021

(54) Feminine Incontinence Apparatus

(71) Applicant: Donetta Davis, Chanute, KS (US)

(72) Inventor: Donetta Davis, Chanute, KS (US)

(21) Appl. No.: 16/705,784

(22) Filed: Dec. 6, 2019

(51) Int. Cl. A61F 13/47; A61F 13/15; A61F 13/534; A61F 13/539; A61F 13/49

(52) U.S. Cl. CPC A61F 13/47; A61F 13/15203; A61F 13/49001; A61F 13/539; A61F 13/534

(57) ABSTRACT

A feminine incontinence apparatus for providing a comfortable and discrete feminine pad includes an absorptive pad comprising a base layer having a base top side, a base bottom side, a base front edge, a base back edge, a base left edge, and a base right edge. A first absorptive layer is coupled to the base top side. The base layer and the first absorptive layer have a gluteal crease extending from the base back edge along a medial axis towards the base front edge. The gluteal crease is configured to fold within a user's intergluteal cleft. The base bottom side is configured to be placed within an undergarment such that the base front edge rests adjacent a user's pubic region and the base back edge rests within the intergluteal cleft.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to feminine incontinence devices and more particularly pertains to a new feminine incontinence device for providing a comfortable and discrete feminine pad.

### (2) Description of Related Art Including Information Disclosed Under 37 CFR 1.97 and 1.98

The prior art relates to feminine incontinence devices.

### BRIEF SUMMARY OF THE INVENTION

An embodiment of the disclosure meets the needs presented above by generally comprising an absorptive pad including a base layer having a base top side, a base bottom side, a base front edge, a base back edge, a base left edge, and a base right edge. A first absorptive layer is coupled to the base top side. The base layer and the first absorptive layer have a gluteal crease extending from the base back edge along a medial axis towards the base front edge. The gluteal crease is configured to fold within a user's intergluteal cleft. The base bottom side is configured to be placed within an undergarment such that the base front edge rests adjacent a user's pubic region and the base back edge rests within the intergluteal cleft.

In another embodiment, the absorptive pad is coupled to an underwear brief. The underwear brief is configured to be worn like a traditional, form-fitting underwear. The underwear brief has a rear side configured to conform within the intergluteal cleft between a user's buttocks.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a feminine incontinence apparatus according to an embodiment of the disclosure.

FIG. 2 is a top plan view of an embodiment of the disclosure.

FIG. 3 is a bottom plan view of an embodiment of the disclosure.

FIG. 4 is a side elevation view of an embodiment of the disclosure.

FIG. 5 is an in-use view of an embodiment of the disclosure.

FIG. 6 is a front elevation view of an embodiment of the disclosure.

FIG. 7 is a front elevation view of an embodiment of the disclosure.

### DETAILED DESCRIPTION OF THE INVENTION

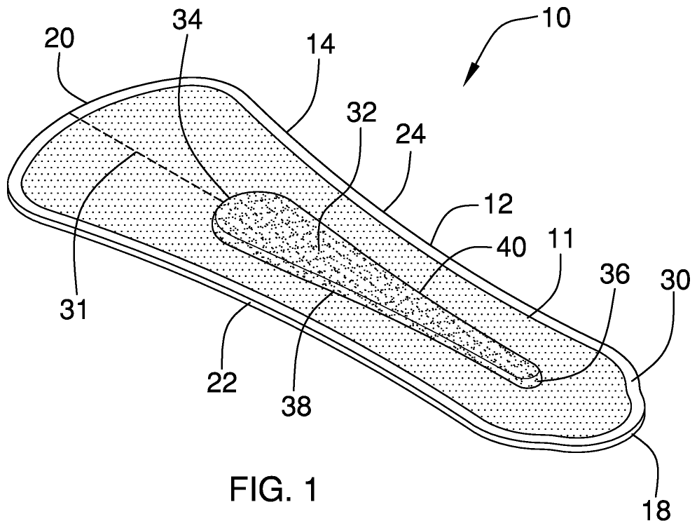

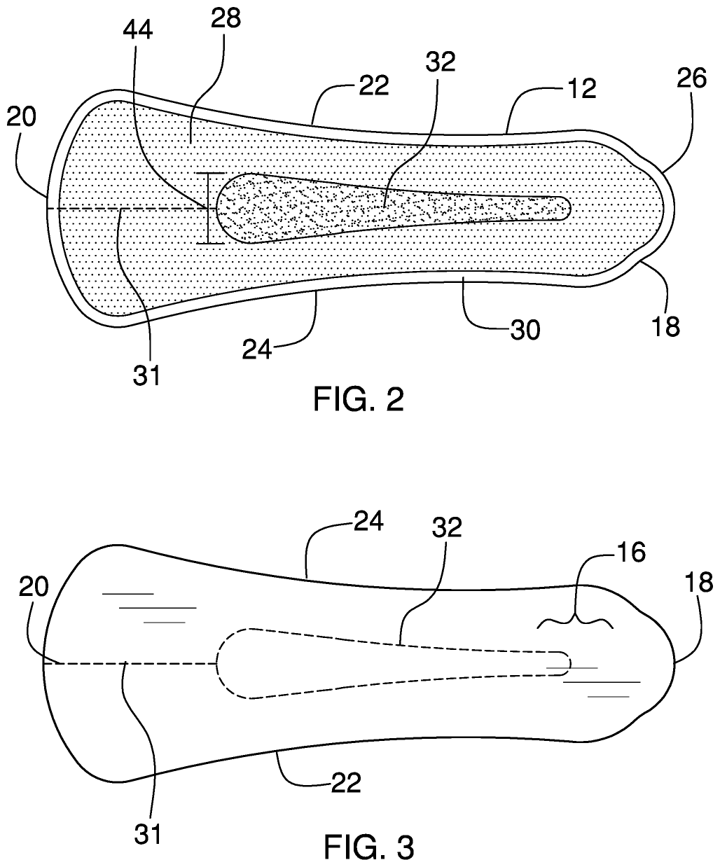

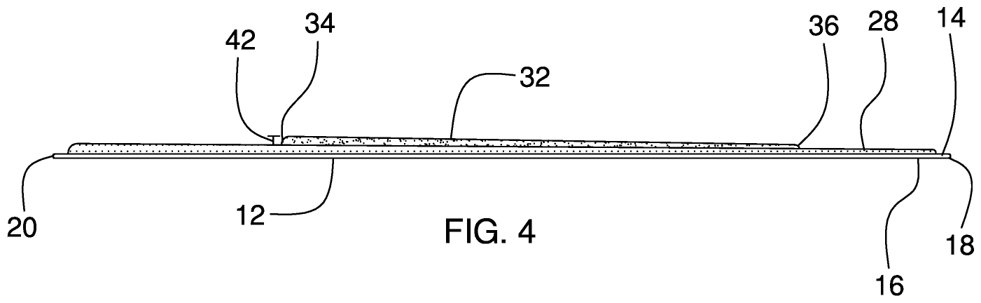

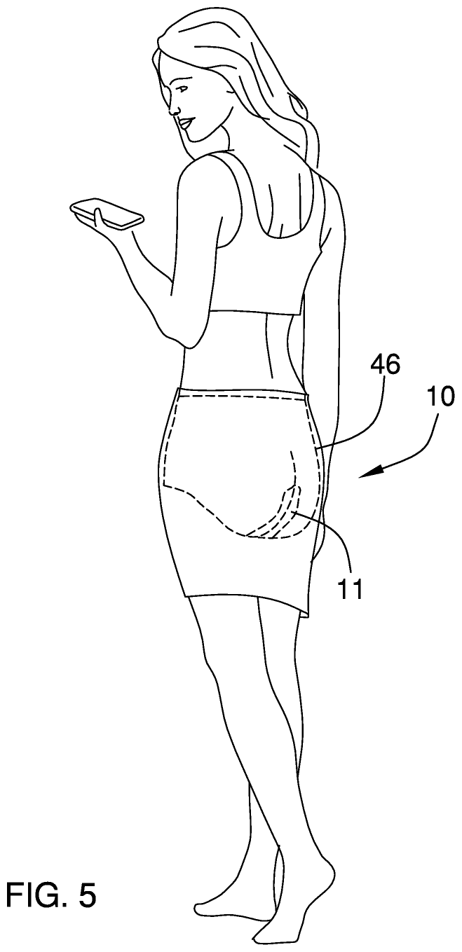

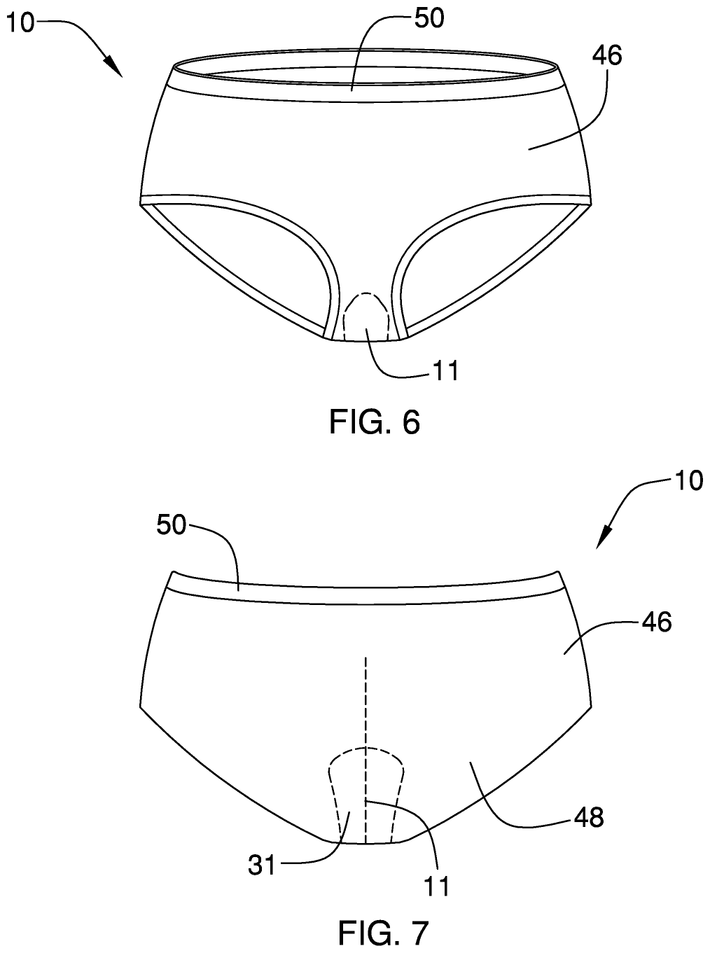

With reference now to the drawings, and in particular to FIGS. 1 through 7 thereof, a new feminine incontinence device embodying the principles and concepts of an embodiment of the disclosure and generally designated by the reference numeral 10 will be described.

As best illustrated in FIGS. 1 through 7, the feminine incontinence apparatus 10 generally comprises an absorptive pad 11 including a base layer 12 having a base top side 14, a base bottom side 16, a base front edge 18, a base back edge 20, a base left edge 22, and a base right edge 24. The base left edge 22 and the base right edge 24 may each be convex relative a medial axis extending from the base front edge 18 to the base back edge 20 in order to comfortably fit between a user's legs. The base front edge 18 may have a semicircular protrusion 26 to maximize coverage area. The base layer 12 may be waterproof. A first absorptive layer 28 is coupled to the base top side 14 of the base layer 12 and conforms to the shape of the base front edge 18, the base back edge 20, the base left edge 22, and the base right edge 24. The first absorptive layer 28 thus defines a perimeter region 30 of the base top side. The perimeter region 30 is uniform and acts as a boundary for the first absorptive layer 28 to prevent leaks. Each of the base layer 12 and the first absorptive layer 28 have a gluteal crease 31 extending from the base back edge 20 along the medial axis towards the base front edge 18. The gluteal crease 31 is configured to fold within a user's intergluteal cleft to be as form fitting as possible.

A second absorptive layer 32 may be coupled to the first absorptive layer 28. The second absorptive layer 32 has a pad front edge 34, a pad back edge 36, a pad left edge 38, and a pad right edge 40. A pad thickness 42 of the second absorptive layer above the first absorptive layer 28 tapers thinner from the pad back edge 36 to the pad front edge 34. A pad width 44 between the pad left edge 38 and the pad right edge 40 also tapers thinner from the pad back edge 36 to the pad front edge 34. The pad front edge 34 may be semicircular.

In another embodiment, the absorptive pad 11 is coupled to an underwear brief 46. The underwear brief 46 is configured to be worn like a traditional, form-fitting underwear. The underwear brief 46 has a rear side 48 configured to conform within the intergluteal cleft between a user's buttocks to create a discrete incontinence device that can be worn with any type of clothing. The base bottom side 16 is coupled within the underwear brief 46 such that the base front edge 18 is configured to rest adjacent a user's pubic region and the base back edge 20 extends up the rear side 48 of the underwear brief within the intergluteal cleft. A waistband 50 of the underwear brief may be configured to be worn adjacent a user's natural waistline.

In use, the apparatus 10 is worn as a traditional feminine incontinence device whether independently or integrated within the underwear brief 46. The gluteal crease 31 folds within the user's intergluteal cleft to allow the apparatus 10 to be more discrete and form fitting than a traditional incontinence device.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A feminine incontinence apparatus comprising:
an absorptive pad comprising a base layer having a base top side, a base bottom side, a base front edge, a base back edge, a base left edge, and a base right edge; and
a first absorptive layer coupled to the base layer, the first absorptive layer being coupled to the base top side, the base layer and the first absorptive layer having a gluteal crease extending from the base back edge along a medial axis towards the base front edge, the gluteal crease being configured to fold within a user's intergluteal cleft, the base bottom side being configured to be placed within an undergarment such that the base front edge rests adjacent a user's pubic region and the base back edge rests within the intergluteal cleft.

2. The feminine incontinence apparatus of claim 1 further comprising the first absorptive layer conforming to the shape of the base front edge, the base back edge, the base left edge, and the base right edge, the first absorptive layer defining a perimeter region of the base top side.

3. The feminine incontinence apparatus of claim 1 further comprising a second absorptive layer coupled to the first absorptive layer, the second absorptive layer having a pad front edge, a pad back edge, a pad left edge, and a pad right edge.

4. The feminine incontinence apparatus of claim 3 further comprising a pad thickness of the second absorptive layer above the first absorptive layer tapering thinner from the pad front edge to the pad back edge.

5. The feminine incontinence apparatus of claim 1 further comprising the base left edge and the base right edge each being convex relative a medial axis extending from the base front edge to the base back edge.

6. The feminine incontinence apparatus of claim 1 further comprising a pad width between the pad left edge and the pad right edge tapering thinner from the pad back edge to the pad front edge.

7. The feminine incontinence apparatus of claim 1 further comprising the base front edge having a semicircular protrusion.

8. A feminine incontinence apparatus comprising:
a base layer, the base layer having a base top side, a base bottom side, a base front edge, a base back edge, a base left edge, and a base right edge, the base left edge and the base right edge each being convex relative a medial axis extending from the base front edge to the base back edge, the base front edge having a semicircular protrusion;
a first absorptive layer coupled to the base layer, the first absorptive layer being coupled to the base top side and conforming to the shape of the base front edge, the base back edge, the base left edge, and the base right edge, the first absorptive layer defining a perimeter region of the base top side, the base layer and the first absorptive layer having a gluteal crease extending from the base back edge along a medial axis towards the base front edge, the gluteal crease being configured to fold within a user's intergluteal cleft, the base bottom side being configured to be placed within an undergarment such that the base front edge rests adjacent a user's pubic region and the base back edge rests within the intergluteal cleft; and
a second absorptive layer coupled to the first absorptive layer, the second absorptive layer having a pad front edge, a pad back edge, a pad left edge, and a pad right edge, a pad thickness of the second absorptive layer above the first absorptive layer tapering thinner from the pad back edge to the pad front edge, a pad width between the pad left edge and the pad right edge tapering thinner from the pad back edge to the pad front edge.

9. A feminine incontinence apparatus comprising:
an underwear brief, the underwear brief being configured to be worn like a traditional, form-fitting underwear, the underwear brief having a rear side configured to conform within an intergluteal cleft between a user's buttocks; and
an absorptive pad coupled to the underwear brief, the absorptive pad comprising:
a base layer, the base layer having a base top side, a base bottom side, a base front edge, a base back edge, a base left edge, and a base right edge, the base bottom side being coupled within the underwear brief such that the base front edge is configured to rest adjacent a user's pubic region and the base back edge extends up the rear side of the underwear brief within the intergluteal cleft; and
a first absorptive layer coupled to the base layer, the first absorptive layer being coupled to the base top side, the base layer and the first absorptive layer having a gluteal crease extending from the base back edge along a medial axis towards the base front edge, the gluteal crease being configured to fold within a user's intergluteal cleft.

10. The feminine incontinence apparatus of claim 9 further comprisinga

11. The feminine incontinence apparatus of claim 9 further comprising the first absorptive layer conforming to the shape of the base front edge, the base back edge, the base left edge, and the base right edge, the first absorptive layer defining a perimeter region of the base top side.

12. The feminine incontinence apparatus of claim 9 further comprising a second absorptive layer coupled to the first absorptive layer, the second absorptive layer having a pad front edge, a pad back edge, a pad left edge, and a pad right edge.

13. The feminine incontinence apparatus of claim 12 further comprising a pad thickness of the second absorptive layer above the first absorptive layer tapering thinner from the pad front edge to the pad back edge.

14. The feminine incontinence apparatus of claim 9 further comprising the base left edge and the base right edge each being convex relative a medial axis extending from the base front edge to the base back edge.

15. The feminine incontinence apparatus of claim 9 further comprising a pad width between the pad left edge and the pad right edge tapering thinner from the pad back edge to the pad front edge.

16. The feminine incontinence apparatus of claim 9 further comprising the base front edge having a semicircular protrusion.
